(19) United States
(12) Patent Application Publication
(10) Pub. No.: US 2009/0324734 A1
(43) Pub. Date: Dec. 31, 2009

(54) THROAT CARE COMPOSITION

(76) Inventor: Daniel Dickey, Westbury, NY (US)

Correspondence Address: AMSTER, ROTHSTEIN & EBENSTEIN LLP, 90 PARK AVENUE, NEW YORK, NY, 10016, US

(21) Appl. No.: 12/147,127

(22) Filed: Jun. 26, 2008

(51) Int. Cl. A61K 35/64; A61P 11/04

(52) U.S. Cl. 424/537

(57) ABSTRACT

A throat care composition including water, honey, vegetable glycerine, natural lemon flavor, at least one of liquefied slippery elm and liquefied marshmallow root, grape seed extract and malic acid. In at least one embodiment, the throat care composition is applied via a spray applicator.

### FIELD OF THE INVENTION

The present disclosure relates to a throat care composition, and more specifically to a throat care composition that improves vocal quality and promotes throat comfort without causing throat numbness.

### BACKGROUND OF THE INVENTION

People working in professions that require extensive use of their voice, such as professional singers and radio announcers, must pay particular attention to the health of their vocal chords to ensure that their voice maintains its proper timbre and to prevent vocal chord damage. In this regard, people in such professions often use medicinal solutions, whether home-made or store-bought, which may be applied to the throat by, for example, a spray or lozenge. These solutions may be effective in moisturizing the user's throat to prevent hoarseness, for example. However, such solutions are typically foul-tasting, and further, often contain ingredients that result in numbing of the throat. Such numbing can result in damage to the user's vocal chords, without the user being aware of the damage.

Accordingly, there is a need for a throat care composition that is pleasant-tasting and that does not result in throat numbness, while also being effective in moisturizing the user's throat and stimulating the production of saliva.

### SUMMARY OF THE INVENTION

A throat care composition according to an exemplary embodiment of the present invention comprises: water; honey; vegetable glycerine; natural lemon flavor; at least one of liquefied slippery elm and liquefied marshmallow root; grape seed extract; and malic acid.

An apparatus for throat care according to an exemplary embodiment of the present invention comprises: a throat care composition comprising: water; honey; vegetable glycerine; natural lemon flavor; at least one of liquefied slippery elm and liquefied marshmallow root; grape seed extract; and malic acid; and a spray bottle for administering the throat care composition to a throat of a user.

A method of forming a throat care product according to an exemplary embodiment of the present invention comprises the step of combining the following ingredients into a throat care composition: water; honey; vegetable glycerine; natural lemon flavor; at least one of liquefied slippery elm and liquefied marshmallow root; grape seed extract; and malic acid.

In at least one embodiment, the water is present in an amount of approximately 6% by weight.

In at least one embodiment, the honey is present in an amount of approximately 30% by weight.

In at least one embodiment, the vegetable glycerine is present in an amount of approximately 40% by weight.

In at least one embodiment, the liquefied slippery elm is present in an mount of approximately 0.175% by weight.

In at least one embodiment, the liquefied marshmallow root is present in an amount of approximately 0.175% by weight.

In at least one embodiment, the malic acid is present in an amount of approximately 1.00% by weight.

In at least one embodiment, the lemon flavor is present in an amount of approximately 4% by weight.

In at least one embodiment, the composition is delivered via a spray applicator.

In at least one embodiment, the composition further comprises one or more of the following ingredients: zinc, Echinacea, Golden Seal, vitamin C, vitamin B-complex, guarana, aloe or hyssop.

These and other features of this invention are described in, or are apparent from, the following detailed description of various exemplary embodiments of this invention.

### BRIEF DESCRIPTION OF THE FIGURES

Various exemplary embodiments of the invention will be described in detail, with reference to the following figures, wherein:

FIG. 1 shows an apparatus for throat care according to an exemplary embodiment of the present invention.

### DETAILED DESCRIPTION OF PREFERRED EMBODIMENTS

The present invention is directed to a throat care composition for soothing sore throats and improving vocal performance. In various exemplary embodiments, the inventive throat care composition includes natural ingredients which have a combined effect of stimulating the production of saliva, as well as lubricating, moisturizing and reducing swelling within the throat. The throat care composition according to the present invention is effective without numbing the vocal folds, which may otherwise cause injury that is not felt by the user until the numbness wears off.

The throat care composition according to an exemplary embodiment of the present invention includes water, honey, vegetable glycerine, natural lemon flavor, at least one of liquefied slippery elm and liquefied marshmallow root, grape seed extract and malic acid. The relative amounts of each of these ingredients and their intended functions within the composition are discussed below.

Water functions as a general lubricant and moisturizer within the composition, and also provides the composition with more fluid-like properties to allow for ease of delivery and application to the throat. Preferably, water is present in the composition in an amount of approximately 6% by weight of composition.

Honey as added to the composition functions as a salivary stimulant. In particular, honey contains relatively large sugar molecules, which are absorbed by the mucous membrane, the lining of the inside of the mouth and throat, thus stimulating the production of saliva. Preferably, honey is present in the composition in an amount of approximately 30% by weight of composition.

Vegetable glycerine as added to the composition functions as a vocal chord lubricant. In particular, vegetable glycerine is instantly absorbed by the mucous membrane from where it then flows to and coats the vocal chords. Preferably, vegetable glycerine is present in the composition in an amount of approximately 40% by weight of composition.

Natural lemon flavor and malic acid, which is a naturally occurring fruit-based acid, also stimulate the production of saliva due to their tartness and the salivary glands reaction to the tartness. Preferably, natural lemon flavor is present in the composition in an amount of approximately 4% by weight of composition, and malic acid is present in the composition in an amount of approximately 1% by weight of composition.

Liquefied slippery elm and liquefied marshmallow root as added to the composition function as emulsants, in that they are naturally hydrophilic and therefore attract moisture to the vocal chords. Preferably, each of the liquefied slippery elm and liquefied marshmallow root is present in the composition in an amount of approximately 0.175% by weight of composition.

Grape seed extract as added to the composition function as an anti-inflammatory, which helps reduce swelling of the vocal chords. Preferably, grape seed extract is present in the composition in an amount of approximately 0.175%.

The following example is provided to show one possible combination of the above-mentioned ingredients:

### Example 1

A throat care composition including the following ingredients:

⅛ oz. water;
0.65 honey;
0.75 oz. vegetable glycerine;
½ tspn natural lemon flavor;
100 mg liquefied slippery elm;
100 mg liquefied marshmallow root;
100 mg grape seed extract;
600 mg malic acid.

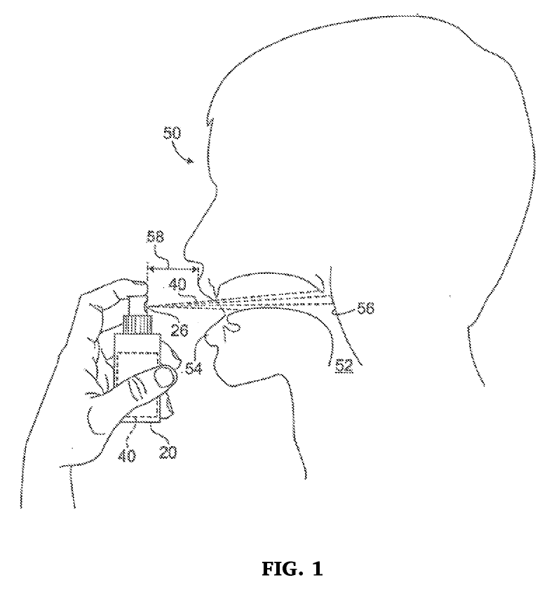

The throat care composition according to various exemplary embodiments of the present invention may be provided within a spray bottle, so that it may be more easily applied to the throat of a user. In particular, as shown in FIG. 1, a user 50 may hold a spray bottle 20 containing the throat care composition 40 a certain distance 58 from the user's mouth 54, and then activate a spray nozzle 26 on the spray bottle 20 to spray the throat care composition 40 onto the back 56 of the user's throat 52. The throat care composition 40 will then naturally flow to and act upon the user's vocal chords. It should be appreciated that the present invention is not limited to a spray, and in other exemplary embodiments the throat care composition may be administered in any suitable form, such as, for example, as a lozenge or a mouth wash.

In various exemplary embodiments, the inventive throat care composition may further include various medicinal ingredients, such as, for example, zinc, Echinacea, Golden Seal, vitamin C, vitamin B complex, guarana, aloe and hyssop.

While the foregoing invention has been described in some detail for purposes of clarity and understanding, it will be appreciated by one skilled in the art from a reading of the disclosure that various changes in form and detail can be made without departing from the true scope of the invention in the appended claims.

What is claimed is:

1. A throat care composition, comprising:
water;
honey;
vegetable glycerine;
natural lemon flavor;
at least one of liquefied slippery elm and liquefied marshmallow root;
grape seed extract; and
malic acid.

2. The throat care composition of claim 1, wherein the water is present in an amount of approximately 6% by weight.

3. The throat care composition of claim 1, wherein the honey is present in an amount of approximately 30% by weight.

4. The throat care composition of claim 1, wherein the vegetable glycerine is present in an amount of approximately 40% by weight.

5. The throat care composition of claim 1, wherein the liquefied slippery elm is present in an mount of approximately 0.175% by weight.

6. The throat care composition of claim 1, wherein the liquefied marshmallow root is present in an amount of approximately 0.175% by weight.

7. The throat care composition of claim 1, wherein the malic acid is present in an amount of approximately 1% by weight.

8. The throat care composition of claim 1, wherein the lemon flavor is present in an amount of approximately 4% by weight.

9. The throat care composition of claim 1, wherein the composition is delivered via a spray applicator.

10. The throat care composition of claim 1, wherein the composition further comprises one or more of the following ingredients: zinc, Echinacea, Golden Seal, vitamin C, vitamin B-complex, guarana, aloe or hyssop.

11. An apparatus for throat care, comprising:
a throat care composition comprising:
water;
honey;
vegetable glycerine;
natural lemon flavor;
at least one of liquefied slippery elm and liquefied marshmallow root;
grape seed extract; and
malic acid; and
a spray bottle for administering the throat care composition to a throat of a user.

12. The apparatus of claim 11, wherein the water is present in an amount of approximately 6% by weight.

13. The apparatus of claim 11, wherein the honey is present in an amount of approximately 30% by weight.

14. The apparatus of claim 11, wherein the vegetable glycerine is present in an amount of approximately 40% by weight.

15. The apparatus of claim 11, wherein the liquefied slippery elm is present in an mount of approximately 0.175% by weight.

16. The apparatus of claim 11, wherein the liquefied marshmallow root is present in an amount of approximately 0.175% by weight.

17. The apparatus of claim 11, wherein the malic acid is present in an amount of approximately 1% by weight.

18. The apparatus of claim 11, wherein the lemon flavor is present in an amount of approximately 4% by weight.

19. The apparatus of claim 11, wherein the composition is delivered via a spray applicator.

20. The apparatus of claim 11, wherein the composition further comprises one or more of the following ingredients: zinc, Echinacea, Golden Seal, vitamin C, vitamin B-complex, guarana, aloe or hyssop.

21. A method of forming a throat care product, comprising the step of combining the following ingredients into a throat care composition:
water;
honey;
vegetable glycerine;
natural lemon flavor;
at least one of liquefied slippery elm and liquefied marshmallow root;
grape seed extract; and
malic acid.

22. The method of claim 21, wherein the step of combining comprises adding the water by an amount of approximately 6% by weight.

23. The method of claim 21, wherein the step of combining comprises adding the honey by an amount of approximately 30% by weight.

24. The method of claim 21, wherein the step of combining comprises adding vegetable glycerine by an amount of approximately 40% by weight.

25. The method of claim 21, wherein the step of combining comprises adding the liquefied slippery elm by an mount of approximately 0.175% by weight.

26. The method of claim 21, wherein the step of combining comprises adding the liquefied marshmallow root by an amount of approximately 0.175% by weight.

27. The method of claim 21, wherein the step of combining comprises adding the malic acid is by an amount of approximately 1% by weight.

28. The method of claim 21, wherein the step of combining comprises adding the lemon flavor to an amount of approximately 4% by weight.

29. The method of claim 21, further comprising the step of providing the throat care composition in a spray bottle.

30. The method of claim 21, further comprising the step of adding to the throat care composition one or more of the following ingredients: zinc, Echinacea, Golden Seal, vitamin C, vitamin B-complex, guarana, aloe or hyssop.
